(19) United States
(12) Patent Application Publication
(10) Pub. No.: US 2016/0232330 A1
(43) Pub. Date: Aug. 11, 2016

(54) Manifold Diffusion of Solutions for Kinetic Analysis of Pharmacokinetic Data

(71) Applicant: Commonwealth Scientific and Industrial Research Organisation, Campbell (AU)

(72) Inventor: Nicholas Delanie Hirst DOWSON, Campbell (AU)

(21) Appl. No.: 15/023,695

(22) PCT Filed: Sep. 26, 2014
(86) PCT No.: PCT/AU2014/000937

§ 371 Date: Mar. 22, 2016

(30) Foreign Application Priority Data
Sep. 27, 2013 (AU) 2013903740

(51) Int. Cl. G06F 19/00; A61B 5/055; A61B 6/00; G01R 33/56; A61B 6/03

(52) U.S. Cl. CPC G06F 19/704; G01R 33/5601; G06F 19/3437; A61B 6/037; A61B 6/032; A61B 6/481; A61B 5/055

(57) ABSTRACT

A method and system enables efficient and robust analysis of pharmacokinetic data. The method includes providing data of a plurality of pharmacokinetic time activity curves (TACs), wherein each pharmacokinetic TAC corresponds to a portion of the pharmacokinetic data; generating a first set of parameters of a pharmacokinetic mode!, the first set of parameters providing a first estimate of kinetic parameters of a first pharmacokinetic TAG of the plurality of pharmacokinetic TACs; and associating the first set of parameters with a second pharmacokinetic TAG of the plurality of pharmacokinetic TACs, wherein the first set of parameters additionally provides a first estimate of kinetic parameters of the second pharmacokinetic TAG.

### FIELD OF THE INVENTION

The present invention relates to analysis of pharmacokinetic data. In particular, although not exclusively, the invention relates to low complexity kinetic analysis of medical imaging data.

### BACKGROUND TO THE INVENTION

Positron emission tomography (PET) is a medical imaging technique in which a radioactive tracer is introduced into the body and a scanner measures a tracer concentration in various parts of the body. Magnetic resonance imaging (MRI) is another medical imaging technique that can be used with a contrast agent for a similar purpose. In such cases, an image is generally formed based upon the data from the scanner, wherein the image illustrates levels of the tracer in the relevant part of the body.

In PET and MRI, a sequence of images is acquired over a period of time. Each image comprises a set of elements, each of which includes at least one intensity value. The intensity values for individual elements typically vary over time, and thus the sequence of images describe time-based changes in an object. Such variation in intensity for an individual element is referred to as a time activity curve (TAC). Analysis of sequences of images can be used to quantify various functions of the body, such as blood flow and perfusion of an organ or tissue, which can in turn be used to identify diseases and disease states.

In particular, kinetic analysis of PET images is important in understanding many biological phenomena such as cancer. Kinetic analysis involves the inverse transformation of a time activity curve (TAC) into a set of kinetic parameters for a defined model and a blood input function (BIF). The kinetic parameters can then be used to characterise the corresponding tissue

Due to the high computational cost of kinetic analysis of PET and MRI images and the noise that is typically present in PET data, kinetic analysis frequently relies on regional analysis of the images. In such case, TACs are generally calculated for a region as a whole, rather than for individual pixels or voxels. As such, all the data within each region is averaged, resulting in a loss of information. Such loss of information can be particularly relevant in the case of cancer, because tumours are known to be highly heterogeneous objects.

Certain attempts have been made to reduce a computational expense of kinetic analysis of PET images by, for example, matching the data to pre-computed dictionaries, or by using simplified models. These approaches, however, are often not robust to noisy data or inadequately represent the underlying biology of individual regions.

Accordingly, there is a need for an improved system and method for kinetic analysis of pharmacokinetic data.

### OBJECT OF THE INVENTION

It is an object of some embodiments of the present invention to provide improvements and advantages over the above described prior art, and/or overcome and alleviate one or more of the above described disadvantages of the prior art, and/or provide a useful commercial choice.

### SUMMARY OF THE INVENTION

According to a first embodiment, the invention resides in a method of analysing pharmacokinetic data comprising:

providing data of a plurality of pharmacokinetic time activity curves (TACs), wherein each pharmacokinetic TAC corresponds to a portion of the pharmacokinetic data;

generating a first set of parameters of a pharmacokinetic model, the first set of parameters providing a first estimate of kinetic parameters of a first pharmacokinetic TAC of the plurality of pharmacokinetic TACs; and

associating the first set of parameters with a second pharmacokinetic TAC of the plurality of pharmacokinetic TACs wherein the first set of parameters additionally provides a first estimate of kinetic parameters of the second pharmacokinetic TAC.

Preferably, the method further comprises:

determining that the first set of parameters provides a better estimate of kinetic parameters of the second pharmacokinetic TAC than an earlier set of parameters associated with the second pharmacokinetic TAC.

Preferably, determining that the first set of parameters provides a better estimate than an earlier set of parameters comprises:

computing a first distance between the second pharmacokinetic TAC and a first simulated TAC defined by the first set of parameters and the model;

computing a second distance between the second pharmacokinetic TAC and a second simulated TAC defined by the earlier set of parameters and the model; and

determining that the second distance is greater than the first distance.

Preferably, providing the plurality of pharmacokinetic TACS comprises:

providing a plurality of images, the plurality of images illustrating concentration of a substance over time; and

generating the plurality of pharmacokinetic TACs from the images.

Preferably, the plurality of images comprises positron emission tomography (PET) images, magnetic resonance imaging (MRI) images or computed tomography images and the substance comprises a radioactive tracer, an MRI contrast agent or a radiocontrast agent.

Preferably, generating the first set of parameters of the pharmacokinetic model comprises performing an update step of an iterative algorithm.

Preferably, the iterative algorithm comprises a gradient search algorithm, a quadratic programming, algorithm, or a stochastic search algorithm. Suitably, the gradient search algorithm comprises a Levenberg-Marquardt algorithm, Powell's algorithm, or a Simplex algorithm. Suitably, the quadratic programming algorithm comprises an interior point algorithm. Suitably, the stochastic search algorithm comprises the simulated annealing algorithm or a stochastic gradient descent algorithm.

For convenience, the distance between a pharmacokinetic TAC and a simulated TAC may be termed the “error”. Preferably the model allows separation of the parameters into those which linearly affect the error and those which non-linearly affect the error; in such cases, the algorithm preferably includes using a non-negative least squares algorithm to find optimal values for the parameters which linearly affect the error and a non-linear optimisation to find optimal values for the parameters which non-linearly affect the error. Preferably, the model includes a blood input function, and first and second compartmental activity functions.

Preferably, the method further comprises:

grouping the plurality of pharmacokinetic TACs; and

sharing the first set of parameters with a group of which the first pharmacokinetic TAC is a member.

Preferably, the grouping is according to a grid.

Preferably, the method further comprises:

performing principle component analysis (PCA) on the plurality of pharmacokinetic time activity curves (TACs);

wherein the grouping is performed in a discretised PCA space.

Preferably, the method further comprises:

determining that an earlier estimate of kinetic parameters of a first pharmacokinetic TAC comprise a poor estimate of the kinetic parameters; and

selecting the first pharmacokinetic TAC for further refinement of the kinetic parameters.

Preferably, computing a distance between a first TAC defined by the first set of parameters and the model is at least an order of magnitude less complex than generating the first set of parameters.

### BRIEF DESCRIPTION OF THE DRAWINGS

To assist in understanding the invention and to enable a person skilled in the art to put the invention into practical effect, preferred embodiments of the invention are described below by way of example only with reference to the accompanying drawings, in which:

FIG. 1 illustrates a method of kinetic analysis of pharmacokinetic data, according to an embodiment of the present invention;

FIG. 2 illustrates a sequence of PET images illustrating a concentration of a tracer over time in a tissue of a human, according to an embodiment of the present invention;

FIG. 3 illustrates a time activity curve illustrating a concentration of a tracer over time, according to an embodiment of the present invention;

FIG. 4 illustrates a two compartment model, according to an embodiment of the present invention;

FIG. 5 illustrates a chart comparing the computational cost in the form of time, for certain embodiments of the method of FIG. 1;

FIG. 6 illustrates a method of kinetic analysis of pharmacokinetic data, according to a further embodiment of the present invention;

FIG. 7 diagrammatically illustrates a computing device, according to an embodiment of the present invention; and

FIG. 8 is an illustration of an exemplary pharmacokinetic analysis, according to an embodiment of the present invention.

Those skilled in the art will appreciate that minor deviations from the layout of components as illustrated in the drawings will not detract from the proper functioning of the disclosed embodiments of the present invention.

### DETAILED DESCRIPTION OF THE INVENTION

Embodiments of the present invention comprise systems and methods for kinetic analysis of pharmacokinetic data. Elements of the invention are illustrated in concise outline form in the drawings, showing only those specific details that are necessary to the understanding of the embodiments of the present invention, but so as not to clutter the disclosure with excessive detail that will be obvious to those of ordinary skill in the art in light of the present description.

In this patent specification, adjectives such as first and second, left and right, front and back, top and bottom, etc., are used solely to define one element or method step from another element or method step without necessarily requiring a specific relative position or sequence that is described by the adjectives. Words such as “comprises” or “includes” are not used to define an exclusive set of elements or method steps. Rather, such words merely define a minimum set of elements or method steps included in a particular embodiment of the present invention.

The reference to any prior art in this specification is not, and should not be taken as, an acknowledgement or any form of suggestion that the prior art forms part of the common general knowledge.

According to one aspect, the invention resides in a method of analysing pharmacokinetic data comprising: providing data of a plurality of pharmacokinetic time activity curves (TACs), wherein each pharmacokinetic TAC corresponds to a portion of the pharmacokinetic data; generating a first set of parameters of a pharmacokinetic model, the first set of parameters providing a first estimate of kinetic parameters of a first pharmacokinetic TAC of the plurality of pharmacokinetic TACs; associating the first set of parameters with a second pharmacokinetic TAC of the plurality of pharmacokinetic TACs, wherein the first set of parameters additionally provides a first estimate of kinetic parameters of the second pharmacokinetic TAC.

Advantages of certain embodiments of the present invention include an ability to efficiently and robustly analyze pharmacokinetic data. In particular, certain embodiments of the present invention enable pharmacokinetic data to be analyzed at low complexity and with high accuracy.

According to certain embodiments, the present invention is particularly suited for large scale data as computational complexity can be scaled sub-linearly with respect to a number of samples.

Advantages of certain embodiments of the present invention include more accurate analysis of pharmacokinetic data as local minima can be avoided.

Furthermore, certain embodiments of the present invention are agnostic to choice of model, blood input function representation and/or optimisation algorithm, enabling widespread use of the invention at a low cost.

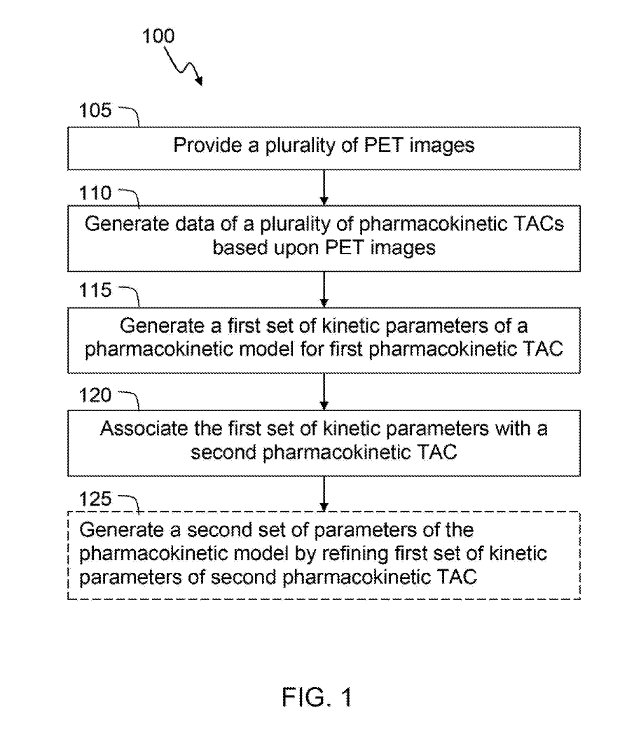

FIG. 1 illustrates a method 100 of kinetic analysis of pharmacokinetic data, according to an embodiment of the present invention.

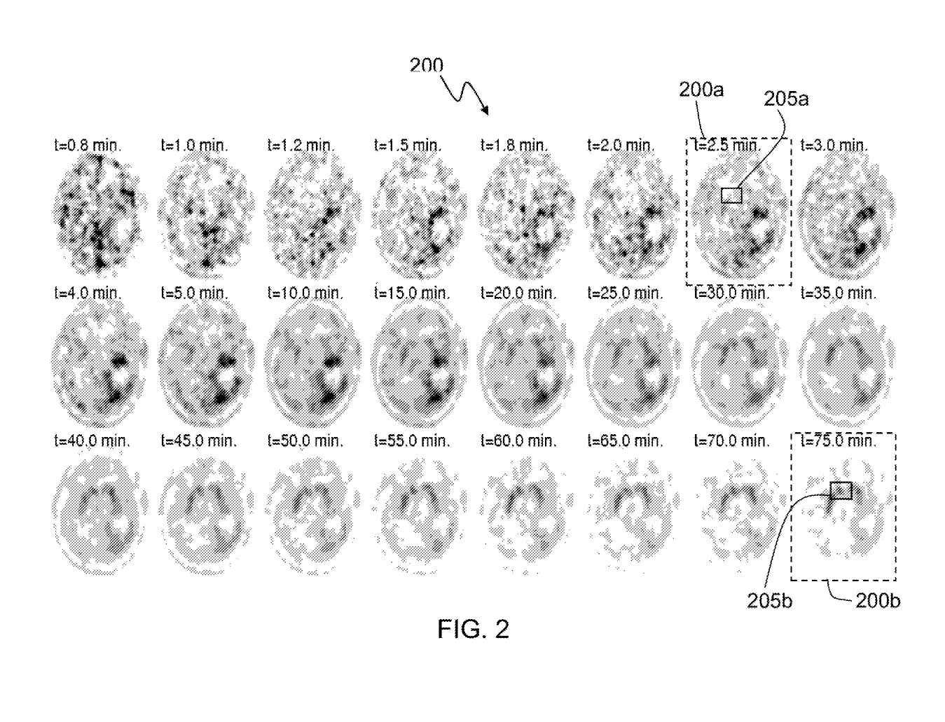

At step 105, a plurality of PET images are provided. The plurality of PET images are one example of pharmacokinetic data and illustrate a concentration of a tracer in, for example, tissue of a human. For example, and referring now to FIG. 2, the plurality of PET images can comprise a sequence of PET images 200 illustrating a concentration of a tracer in a tissue of a human over time. In particular, a first PET image 200a of the sequence of PET images 200 includes a first region 205a of low concentration of the tracer, and a second PET image 200b of the sequence of PET images 200 includes a second region 205b of high concentration of the tracer, wherein the first region 205a and the second region 205b correspond to a common volume of tissue at different time points.

The skilled addressee will readily appreciate that other types of pharmacokinetic data that can be used with the method 100 include Magnetic Resonance Imaging (MRI) and Computed Tomography (CT) data.

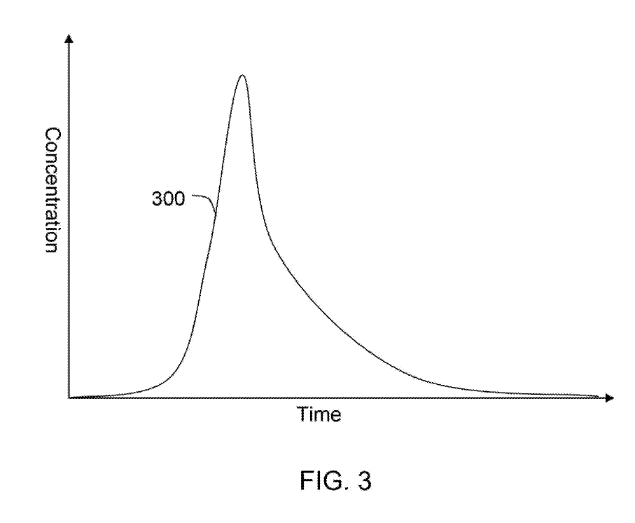

At step 110, data of a plurality of pharmacokinetic time activity curves (TACs) are generated based upon the plurality of PET images. For example, and referring now to FIG. 3, the plurality of pharmacokinetic TACs can include a time activity curve 300 illustrating a concentration of tracer over time.

The plurality of pharmacokinetic TACs are generated by defining a discrete grid of locations in each of the PET images, wherein each pharmacokinetic TAC is a vector of intensities describing the PET uptake for one of the discrete locations across a set of time separated images.

As an illustrative example, the discrete grid of locations comprises pixels of the PET images, such that the pharmacokinetic TAC corresponds to a common pixel in a sequence of PET images. Alternatively, the discrete grid locations can comprise groups of pixels such as the first region 205a and the second region 205b of FIG. 2.

As will be readily understood by the skilled addressee, the pharmacokinetic TACs can be generated according to alternative methods, or provided as input to the method from an unknown source.

At step 115, a first set of parameters of a pharmacokinetic model is generated. The first set of parameters provides a first estimate of kinetic parameters associated with a first pharmacokinetic TAC of the plurality of pharmacokinetic TACs.

As discussed below, the first estimate can be estimated using a first step of an iterative optimisation algorithm configured to estimate the first pharmacokinetic TAC. This enables efficient use of processing resources, as it may not ultimately be necessary to perform further iterations of the optimisation algorithm with respect to the first pharmacokinetic TAC.

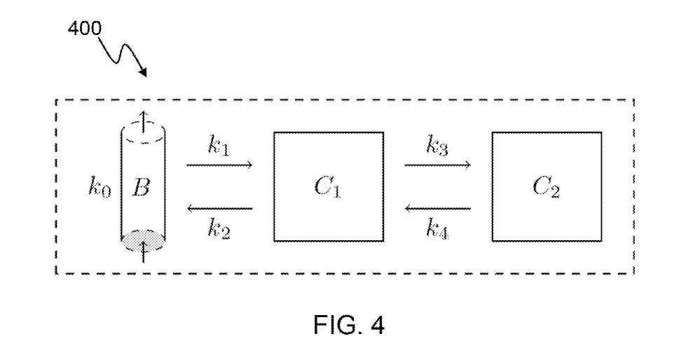

Several pharmacokinetic models are suitable, of which one example is a two compartment model. For example, and referring now to FIG. 4, the model can comprise a two compartment model 400. In such case, the two compartment model 400 comprises a blood input function B, a first compartment C₁ and a second compartment C₂. A plurality of parameters, k₀-k₄ define a relationship between the blood input function B and the first compartment C₁, and between the first compartment C₁ and the second compartment C₂.

The kinetic parameters k₀-k₄ can be solved for a pharmacokinetic TAC using the following equation:

argₖ min D(T(t),S(t;k)) (Equation 1)

wherein:

T(t) is the pharmacokinetic TAC;

k is a vector of the plurality of parameters, k₀-k₄

S(t; k) is a simulation TAC according to the model and the plurality of parameters; and

D is function for determining a distance between TACs.

As will be readily understood by the skilled addressee, Equation 1 can be solved using an iterative algorithm. An example of an iterative algorithm that can be used is the Levenberg-Marquardt algorithm. In such case, the first set of parameters of the pharmacokinetic model can be generated by using one, or a limited number of iterations of the Levenberg-Marquardt algorithm.

As discussed above, the pharmacokinetic model takes the kinetic parameters in the vector k as input. For example, in an ordinary differential equation two compartment formulation, a diagonal matrix, K, can be generated from k, and the blood input function B can be concatenated with a vector of the compartments C₁, C₂ into a vector C as follows:

$$\frac{d}{d t} ( \begin{matrix}{C}_{1} \\ {C}_{2}\end{matrix} ) = K \cdot C = ( \begin{matrix}{k}_{1} & - {k}_{2} - {k}_{3} & {k}_{4} \\ 0 & {k}_{3} & - {k}_{4}\end{matrix} ) \cdot ( \begin{matrix}B \\ {C}_{1} \\ {C}_{2}\end{matrix} ) \qquad ( Equation 2 )$$

Equation 2 can be solved using conventional numerical methods.

The pharmacokinetic model can be formulated in other ways, such as by formulating a weighted sum of exponentials, wherein the simulated TAC can be computed as a convolution of the exponentials.

Similarly, the two compartment model 400 of FIG. 4 can be modified to become a one compartment model by removing the parameters associated with the second compartment C2, or by setting them to zero.

At step 120, the first set of parameters is associated with a second pharmacokinetic TAC of the plurality of pharmacokinetic TACs. The first set of parameters additionally provides a first estimate of kinetic parameters of the second pharmacokinetic TAC.

According to certain embodiments, once the first set of parameters is generated for the first pharmacokinetic TAC, the first set of parameters is compared with parameters of other pharmacokinetic TACs of the plurality of pharmacokinetic TACs. This can be done by calculating a distance between a simulation TAC according to the first set of parameters and the model, and one or more other pharmacokinetic TACs. This distance can then be compared to a previous estimate of the parameters associated with the respective pharmacokinetic TAC.

If the first set of parameters provides a better estimate of any of the other TACs, i.e. the simulation TAC is closer to one or more of the other pharmacokinetic TACs than previous simulation TACs, the first set of parameters is used as new parameters for the one or more other pharmacokinetic TACs.

The second pharmacokinetic TAC, or each of the one or more of the other pharmacokinetic TACs, can then be refined using the first set of parameters as a starting point in step 125.

In step 125, a second set of parameters of the pharmacokinetic model is generated by refining the first estimate of kinetic parameters of the second pharmacokinetic TAC. This step 125 is illustrated in FIG. 1 using dashed lines to emphasise that it is entirely optional and is not included in all embodiments.

By refining the first estimate of kinetic parameters of the second pharmacokinetic TAC, embodiments of the present invention are able to more efficiently determine parameters of the second pharmacokinetic TAC.

Preferably, a simulated TAC associated with the second set of parameters is then compared with other pharmacokinetic TACs of the plurality of pharmacokinetic TACs, and used where the second set of parameters provides a better estimate of the respective pharmacokinetic TAC than an earlier estimate. This process can be repeated several times, wherein each time further refinements of the estimated parameters are made.

Typically, a computational cost associated with computing a distance between a first TAC and a second TAC is at least an order of magnitude less than generating kinetic parameters associated with a TAC. Accordingly, substantial computational savings can be made by comparing new simulation data with existing simulation data of other pharmacokinetic TACs, and sharing the new simulation data among several pharmacokinetic TACs.

Certain formulations of the two compartment model are of a type that include a subset of parameters that have a linear effect upon the error of the estimate and a subset that have a nonlinear effect. In such cases a least squares (preferably a non-negative least squares) algorithm may be used to optimise some or all of the linear parameters, with the remaining parameters being optimised by the iterative algorithm. Such an approach may result in a further reduction in computational complexity.

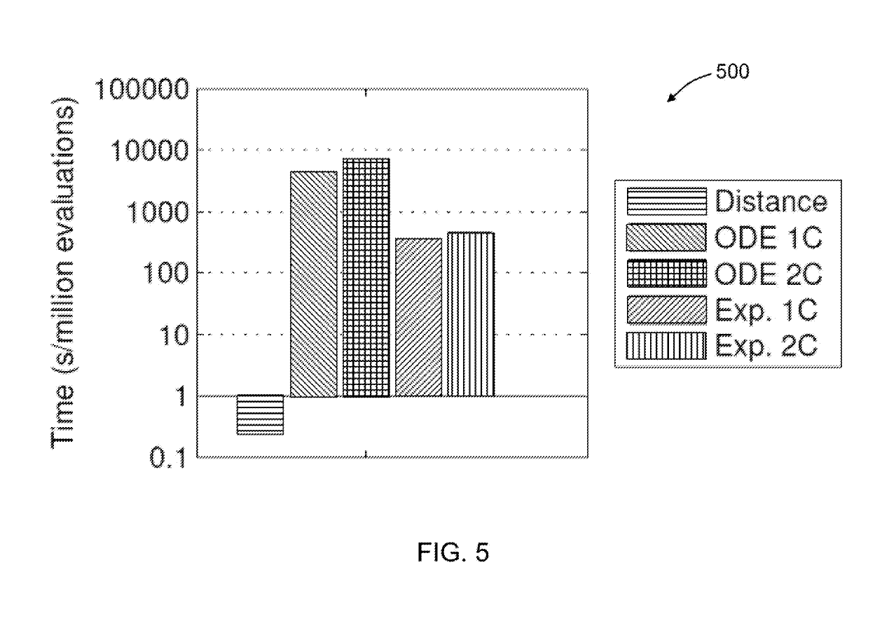

FIG. 5 illustrates a chart 500 comparing the computational cost in the form of time, for different aspects of the method 100 of FIG. 1.

The “distance” evaluation relates to the generation of a similarity measure between two TACs and is based upon a sum of squared differences (SSD) calculation. “ODE 2C” denotes an ordinary differential equation (ODE) two compartment formulation, as described above, and “ODE 1C” denotes an ODE single compartment formulation. “Exp 2C” denotes an exponential two compartment formulation, as described above, and “Exp 1C” denotes an exponential single compartment formulation. The computational costs for the ODE/exponential single and two compartment formulation are evaluated using 26 time-points, e.g. from 26 sequential PET images.

As will be readily understood by a person of ordinary skill in the art, at least 10⁵ data samples can be contained by an image on which kinetic analysis is being performed. As such, substantial data redundancy is often present in the data samples, which enables the method 100 to operate efficiently and robustly.

According to certain embodiments, the method 100 further includes grouping of the plurality of pharmacokinetic TACs. In such case, a nearest neighbour graph can be generated by grouping (for example, by discretising) the space of pharmacokinetic TACs into a set of cells defining a grid. As will be clear to those skilled in the art, the time activity curves can also be grouped using techniques such as hierarchical clustering, centroid based clustering, distribution based clustering or density based clustering.

In such case the parameters associated with one pharmacokinetic TAC, such as the first estimate of kinetic parameters associated with the first pharmacokinetic TAC, can be shared with all neighbouring pharmacokinetic TACs, along with any state data, for further optimisation. Thus grouping of the plurality of pharmacokinetic TACs can provide an inexpensive tool enabling new simulations to be shared between pharmacokinetic TACs. According to certain embodiments, the pharmacokinetic TACs are grouped in a reduced dimensionality space. In such case, principle component analysis (PCA) can be used to reduce dimensionality of the space by performing a singular value decomposition of a covariance matrix of the pharmacokinetic TACs. Eigenvectors of the pharmacokinetic TACs are then ranked in decreasing magnitude, and a subset of eigenvectors is chosen by discarding eigenvectors having a low value.

The PCA space can then be discretised into a set of cells designed to ensure a uniform distribution of samples, as discussed above. As will be clear to those skilled in the art, other dimensionality reduction techniques such as Independent Component Analysis can be used for this step.

According to certain embodiments, the method 100 is initialised with a plurality of kinetic parameters, the initial kinetic parameters referred to as seeds. In such case, enough seeds should be used to ensure “folds” in the manifold are represented, where folds are regions where different kinetic parameters give rise to very similar time activity curves. In these areas, an initial set of parameters may be suboptimal for a given TAC Furthermore, at least some of the seeds should be representative of the data to avoid bias.

As will be readily understood by the skilled addressee, a variety of methods may be used to generate seeds including sampling kinetic parameters on a discrete grid, random sampling, or by simply using a single seed.

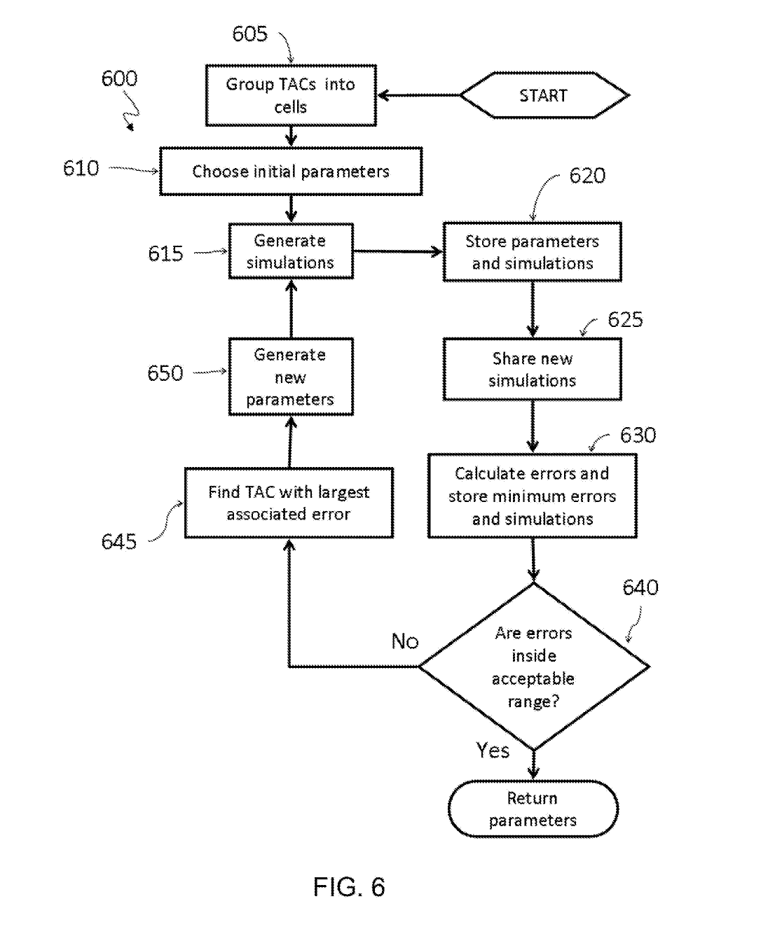

FIG. 6 illustrates a method 600 of kinetic analysis of pharmacokinetic data, according to a further embodiment of the present invention. The method 600 includes analysis of a plurality of pharmacokinetic time activity curves (TACs), each of which can, for example, be generated based upon a plurality of PET images, as discussed above.

In step 605, the plurality of pharmacokinetic TACs are grouped into cells. The cells comprise a discretised space over possible TACs, and can be obtained by principal component analysis of the plurality of TACs, or by any other suitable means.

In step 610, one or more initial sets of kinetic parameters are generated. The one or more initial sets of kinetic parameters can, for example, be generated for a first TAC of the plurality of pharmacokinetic TACs.

In step 615, a simulated TAC is generated for each of the new kinetic parameters, and the simulated TACs and the associated parameters are stored in step 620. According to certain embodiments, the simulated TACs are associated with corresponding cells and shared within a cell or between neighbouring cells.

In step 625, the simulated TACs and associated parameters are shared with at least one other TAC. According to certain embodiments, the simulated TACs are shared with all TACs in the same cell, or with TACs in neighbouring cells.

In step 630, errors are calculated as distances between the TACs and the simulated TACs, and the best matching simulated TACs are associated with each TAC. In particular, an error between each TAC within a cell and each new simulation in the cell is computed. Each TAC has an associated smallest error and corresponding simulation, which can be updated if a new simulated TAC provides a smaller error. In particular, if an error between a TAC and a new simulation is less than the associated smallest error, then the simulation corresponding to the smallest error is replaced with the new simulation and the smallest error is updated.

In this way, after each iteration, each TAC is associated with a best match simulated TAC, and corresponding kinetic parameters and error. Initially the error is set to a very high value to ensure that a new simulation is chosen in the first iteration.

As will be readily understood by the skilled addressee, each TAC need only be compared with a simulated TAC once. In other words, during each iteration, errors need only be calculated for new simulated TACs that were generated during the iteration.

In step 640, it is determined if the errors of all TACs of the plurality of TACs are inside an acceptable range. This can comprise determining if the errors are sufficiently low, e.g. below a threshold, or if any new parameters generated are likely to be too similar to existing parameters.

If the errors of at least some of the TACs of the plurality of TACs are outside of the acceptable range, it is determined which TAC or TACs have the largest errors in step 645. This can be performed by ordering the TACs based upon error, and selecting one or more TACs from the ordered list.

By selecting TACs in which the error is largest, the method 600 ensures that each calculation is performed where it is needed most. As will be clear to those skilled in the art, TACs can also be selected randomly, based on a round-robin scheme, or based on the extent of previous reductions in error, or some other at least one criteria to ensure different TACs are selected and provide reductions in error.

In step 650, new parameters are generated for the selected TACs. A simulated TAC is then generated for each of the new parameters in step 615, as discussed above. The steps described above are then repeated, as illustrated in FIG. 6.

If the best match distances of the TACs are inside of the acceptable range, the parameters are returned. As discussed above, the parameters can be used to quantify various functions of the body, such as blood flow and perfusion of an organ or volume of tissue, which can in turn be used to identify several diseases and disease states.

Under certain circumstances, not all TACs will be associated with simulation data. Accordingly, certain embodiments of the present invention include an additional step of checking for cells or TACs which are not associated with simulation data. In such cases, the TACs which are not associated with simulation data are randomly populated with an existing simulation before further iterations are made.

The above steps are repeated until a predefined number of iterations have been performed, or the improvement, i.e. reduction in total error, falls below a predefined limit.

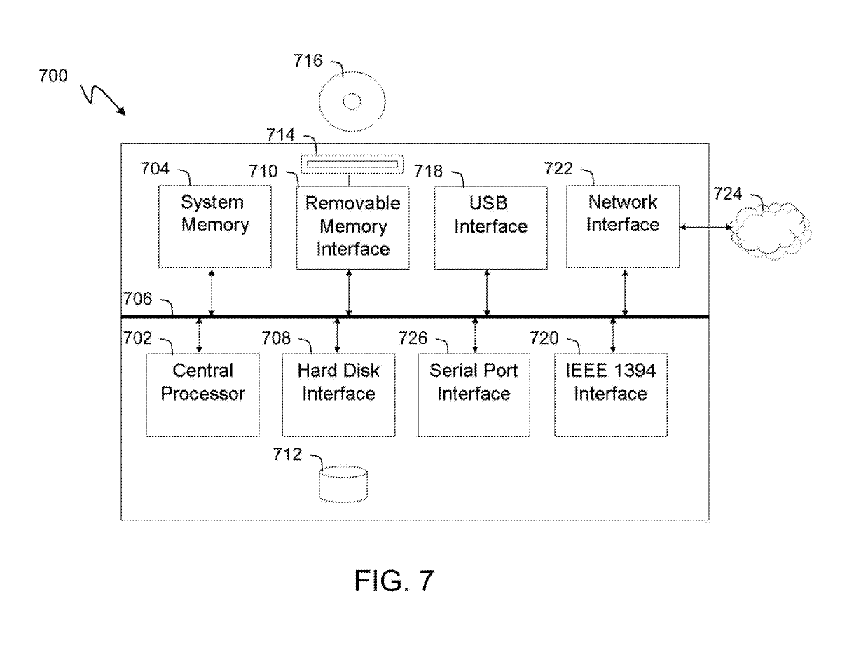

FIG. 7 diagrammatically illustrates a computing device 700, according to an embodiment of the present invention. The method 100 of FIG. 1 can be implemented using the computing device 700.

The computing device 700 includes a central processor 702, a system memory 704 and a system bus 706 that couples various system components, including coupling the system memory 704 to the central processor 702. The system bus 706 may be any of several types of bus structures including a memory bus or memory controller, a peripheral bus, and a local bus using any of a variety of bus architectures. The structure of system memory 704 is well known to those skilled in the art and may include a basic input/output system (BIOS) stored in a read only memory (ROM) and one or more program modules such as operating systems, application programs and program data stored in random access memory (RAM).

The computing device 700 can also include a variety of interface units and drives for reading and writing data. The data can include, for example, concentrations of ingredients, parameters associated with flavour models, and any other associated data.

In particular, the computing device 700 includes a hard disk interface 708 and a removable memory interface 710, respectively coupling a hard disk drive 712 and a removable memory drive 714 to the system bus 706. Examples of removable memory drives 714 include magnetic disk drives and optical disk drives. The drives and their associated computer-readable media, such as a Digital Versatile Disc (DVD) 716 provide non-volatile storage of computer readable instructions, data structures, program modules and other data for the computer system 700. A single hard disk drive 712 and a single removable memory drive 714 are shown for illustration purposes only and with the understanding that the computing device 700 can include several similar drives. Furthermore, the computing device 700 can include drives for interfacing with other types of computer readable media.

The computing device 700 may include additional interfaces for connecting devices to the system bus 706. FIG. 7 shows a universal serial bus (USB) interface 718 which may be used to couple a device to the system bus 706. For example, an IEEE 1394 interface 720 may be used to couple additional devices to the computing device 700. Examples of additional devices include cameras for receiving images or video, or microphones for recording audio.

The computing device 700 can operate in a networked environment using logical connections to one or more remote computers or other devices, such as a server, a router, a network personal computer, a peer device or other common network node, a wireless telephone or wireless personal digital assistant. The computing device 700 includes a network interface 722 that couples the system bus 706 to a local area network (LAN) 724. Networking environments are commonplace in offices, enterprise-wide computer networks and home computer systems.

A wide area network (WAN), such as the Internet, can also be accessed by the computing device, for example via a modem unit connected to a serial port interface 726 or via the LAN 724.

It will be appreciated that the network connections shown and described are exemplary and other ways of establishing a communications link between computers can be used.

The operation of the computing device 700 can be controlled by a variety of different program modules. Examples of program modules are routines, programs, objects, components, and data structures that perform particular tasks or implement particular abstract data types. The present invention may also be practiced with other computer system configurations, including hand-held devices, multiprocessor systems, microprocessor-based or programmable consumer electronics, network PCs, minicomputers, mainframe computers, personal digital assistants and the like. Furthermore, the invention may also be practiced in distributed computing environments where tasks are performed by remote processing devices that are linked through a communications network. In a distributed computing environment, program modules may be located in both local and remote memory storage devices.

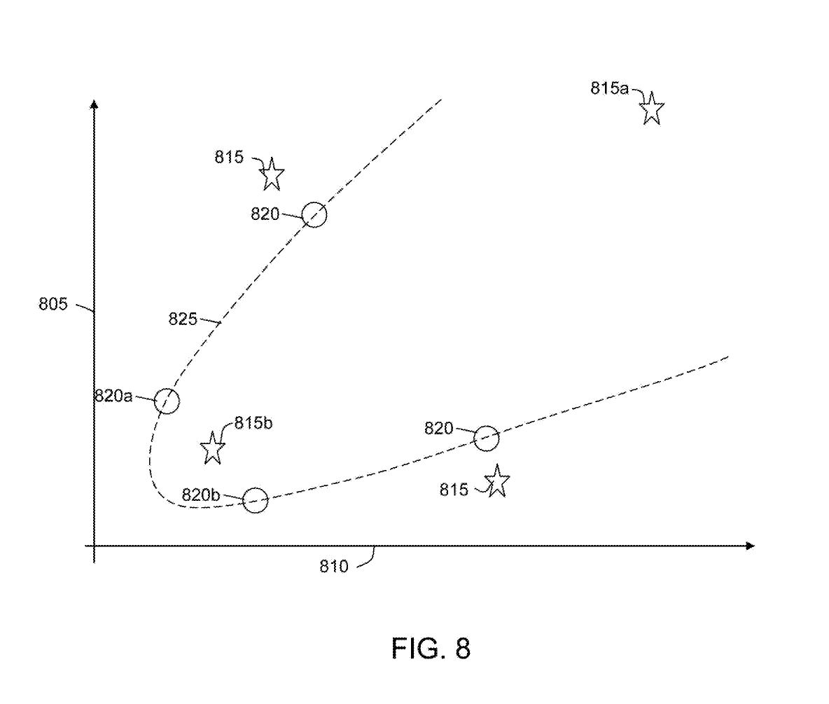

FIG. 8 is an illustration of an exemplary pharmacokinetic analysis, according to an embodiment of the present invention.

A first axis 805 and a second axis 810 define a space on which a plurality of data time activity curves 815, generated from a plurality of images, can be represented. A biological model can be used to generate simulated TACs 820, which lie on a sub-space within the space defined by the first axis 805 and the second axis 810, such subspace illustrated by a dashed line 825.

A search for parameters that define simulated time activity curves 820 is performed, and in particular a search for simulated time activity curves 820 that are a close match to the data time activity curves 815. As discussed above, the subspace is not initially known, and it is computationally expensive to discover a full extent of the subspace. Accordingly, embodiments of the present invention share representations of the subspace. Such sharing enables efficient ways to discover and describe enough of the subspace to generate parameters of the biological model that define simulated time activity curves 820 that lie within a given tolerance of the data time activity curves 815.

The data time activity curves 815 generally form a cloud around the dashed line 825, and many of the data time activity curves 815 do not lie on the dashed line 820 due to noise-induced errors in the data time activity curves 815. In some cases, data time activity curves 815 may be present for which the model is a poor representation, and thus these can be discarded as outliers, as illustrated by an outlier time activity curve 815a. In other cases, several equally appropriate simulated time activity curves 820 may exist for a given data time activity curve 815, as illustrated by time activity curve 815b and simulated time activity curves 820a, 820b.

As will be readily understood by the skilled addressee, time activity curves can be considered as points in a multidimensional space, wherein one dimension is used for each timeframe in which the time activity curves are defined. For the sake of clarity, the illustration of FIG. 8 includes only two dimensions, whereas in practice a large number of dimensions may be required.

In summary, advantages of certain embodiments of the present invention include an ability to efficiently and robustly analyze pharmacokinetic data. In particular, certain embodiments of the present invention enable pharmacokinetic data to be analyzed at low complexity and with high accuracy.

According to certain embodiments, the present invention is particularly suited for large scale application of data as computational complexity can be scaled sub-linearly with respect to a number of samples.

Further advantages of certain embodiments of the present invention include more accurate analysis of pharmacokinetic data as local minima can be avoided.

Furthermore, certain embodiments of the present invention are agnostic to choice of model, blood input function representation and/or optimisation algorithm, enabling widespread use of the invention at a low cost.

The above description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. Accordingly, this patent specification is intended to embrace all alternatives, modifications and variations of the present invention that have been discussed herein, and other embodiments that fall within the spirit and scope of the above described invention.

The claims defining the invention are:

1. A method of analysing pharmacokinetic data, the method comprising:
providing data of a plurality of pharmacokinetic time activity curves (TACs), wherein each pharmacokinetic TAC corresponds to a portion of the pharmacokinetic data;
generating a first set of parameters of a pharmacokinetic model, the first set of parameters providing a first estimate of kinetic parameters of a first pharmacokinetic TAC of the plurality of pharmacokinetic TACs; and
associating the first set of parameters with a second pharmacokinetic TAC of the plurality of pharmacokinetic TACs, wherein the first set of parameters additionally provides a first estimate of kinetic parameters of the second pharmacokinetic TAC.

2. The method of claim 1, further comprising:
determining that the first set of parameters provides a better estimate of kinetic parameters of the second pharmacokinetic TAC than an earlier set of parameters associated with the second pharmacokinetic TAC.

3. The method of claim 2, wherein determining that the first set of parameters provides a better estimate than an earlier set of parameters comprises:
computing a first distance between the second pharmacokinetic TAC and a first simulated TAC defined by the first set of parameters and the model;
computing a second distance between the second pharmacokinetic TAC and a second simulated TAC defined by the earlier set of parameters and the model; and
determining that the second distance is greater than the first distance.

4. The method of claim 1, wherein providing the plurality of pharmacokinetic TACs comprises:
providing a plurality of images, the plurality of images illustrating concentration of a substance over time; and
generating the plurality of pharmacokinetic TACs from the images.

5. The method of claim 4, wherein the plurality of images comprises positron emission tomography (PET) images, magnetic resonance imaging (MRI) images or computed tomography images, and the substance comprises a radioactive tracer, an MRI contrast agent or a radiocontrast agent.

6. The method of claim 1, wherein generating the first set of parameters of the pharmacokinetic model comprises performing an update step of an iterative algorithm.

7. The method of claim 6, wherein the iterative algorithm comprises a gradient search algorithm or a quadratic programming algorithm or a stochastic search algorithm.

8. The method of claim 1, wherein the model includes a blood input function, and first and second compartmental activity functions.

9. The method of claim 1, further comprising:
grouping the plurality of pharmacokinetic TACs into a plurality of groups; and
sharing the first set of parameters with a group of which the first pharmacokinetic TAC is a member.

10. The method of claim 9, wherein the grouping is according to a grid.

11. The method of claim 9, further comprising:
performing principle component analysis (PCA) on the plurality of pharmacokinetic TACs;
wherein the grouping is performed in a discretised PCA space.

12. The method of claim 1, further comprising:
determining that an earlier estimate of kinetic parameters of a first pharmacokinetic TAC comprises a poor estimate of the kinetic parameters; and
selecting the first pharmacokinetic TAC for further refinement of the kinetic parameters.

13. The method of claim 1, further comprising:
refining the kinetic parameters of the plurality of pharmacokinetic TACs by iteratively:
selecting a pharmacokinetic TAC of the plurality of pharmacokinetic TACs;
refining parameters of the pharmacokinetic model associated with the pharmacokinetic TAC; and
associating the refined parameters with at least one other pharmacokinetic TAC.

14. The method of claim 1, wherein computing a distance between a first TAC defined by the first set of parameters and the model is at least an order of magnitude less complex than generating the first set of parameters.

15. The method of claim 1, further comprising generating a second set of parameters of the pharmacokinetic model by refining the first estimate of kinetic parameters of the second pharmacokinetic TAC.

16. A system for analysing pharmacokinetic data, the system comprising:
a processor; and
a memory coupled to the processor, the memory including instruction code executable by the processor for:
generating a first set of parameters of a pharmacokinetic model, the first set of parameters providing a first estimate of kinetic parameters of a first pharmacokinetic TAC of a plurality of pharmacokinetic pharmacokinetic time activity curves (TACs), wherein each pharmacokinetic TAC of the plurality of pharmacokinetic TACs corresponds to a portion of the pharmacokinetic data; and
associating the first set of parameters with a second pharmacokinetic TAC of the plurality of pharmacokinetic TACs, wherein the first set of parameters additionally provides a first estimate of kinetic parameters of the second pharmacokinetic TAC.

17. The system of claim 16, wherein the memory further includes instruction code executable by the processor for:
determining that the first set of parameters provides a better estimate of kinetic parameters of the second pharmacokinetic TAC than an earlier set of parameters associated with the second pharmacokinetic TAC.

18. The system of claim 17, wherein determining that the first set of parameters provides a better estimate than an earlier set of parameters comprises:
computing a first distance between the second pharmacokinetic TAC and a first TAC defined by the first set of parameters and the model;
computing a second distance between the second pharmacokinetic TAC and a second TAC defined by the earlier set of parameters and the model; and
determining that the second distance is greater than the first distance.

19. The system of claim 16, further comprising:
a data interface coupled to the processor;
wherein the memory includes instruction code for receiving positron emission tomography (PET) images; and
wherein the pharmacokinetic data comprises the PET images.

20. The system of claim 16, wherein the memory further includes instruction code executable by the processor for:
determining that an earlier estimate of kinetic parameters of a first pharmacokinetic TAC comprises a poor estimate of the kinetic parameters; and
selecting the first pharmacokinetic TAC for further refinement of the kinetic parameters.

21. The system of claim 16, wherein the memory further includes instruction code executable by the processor for:
refining the kinetic parameters of the plurality of pharmacokinetic TACs by iteratively:
selecting a pharmacokinetic TAC of the plurality of pharmacokinetic TACs;
refining parameters of the pharmacokinetic model associated with the pharmacokinetic TAC; and
associating the refined parameters with at least one other pharmacokinetic TAC.
